(19) United States
(12) Patent Application Publication
(10) Pub. No.: US 2020/0229907 A1
(43) Pub. Date: Jul. 23, 2020

(54) Saliva Ejector Device

(71) Applicant: Heidi Duehlmeier, Salt Lake City, UT (US)

(72) Inventor: Heidi Duehlmeier, Salt Lake City, UT (US)

(21) Appl. No.: 16/840,199

(22) Filed: Apr. 3, 2020

Related U.S. Application Data
(62) Division of application No. 15/286,552, filed on Oct. 5, 2016.
(60) Provisional application No. 62/237,401, filed on Oct. 5, 2015.

(51) Int. Cl. A61C 17/08; A61C 1/00; A61M 1/00

(52) U.S. Cl. CPC A61C 17/08; A61M 1/008; A61M 1/0045; A61C 1/0038

(57) ABSTRACT

A saliva ejector device comprises a handle having a passageway in fluid communication between a vacuum device and a saliva ejector. The saliva ejector device comprises an automatic switching mechanism comprising a button that is switchable between open and closed positions to allow or restrict restricts airflow. The button can be automatically actuatable, via a compliant device in the handle, when an individual places the handle in the cradle or removes the handle from the cradle. The compliant device can be a tube that allows or restricts airflow about the passageway, or a spring in the passageway that actuates the button. A saliva ejector device comprises a handle having an upper housing rotatably coupled to a lower housing to selectively switch the handle between open and closed positions to allow or restrict airflow about the handle. A handle adapter has a spherical body for an individual to cup or hold to improve ergonomics. Associated systems and methods are provided.

### RELATED APPLICATIONS

This is a divisional application of U.S. application Ser. No. 15/286,552, filed Oct. 5, 2016, entitled “Saliva Ejector Device” which claims the benefit of U.S. Provisional Application Ser. No. 62/237,401, filed Oct. 5, 2015, and entitled, “Saliva Ejector Device” each of which is incorporated by reference in its entirety herein.

### BACKGROUND

During dental and oral surgical procedures, a patient is typically in a supine or semi-supine position, which can cause saliva to accumulate in the mouth. This saliva is difficult to swallow because of the patient's position, more particularly because the patient's mouth is open for extended periods of time to facilitate the dental or oral procedures. Further, blood, bacteria, and/or foreign matter may accumulate as a result of the dental or surgical procedures. The removal of water, saliva and foreign matter by means of a vacuum, from a patient's mouth, are widely used in dental, orthodontic and other similar offices where oral procedures are performed. Dental and similar procedures often require repeated use of the vacuum systems to extract the water, saliva and foreign matter. As they are currently configured, bacteria and other microbes tend to collect on saliva ejector housings/handles, which is exacerbated by the fact that such handles often have externally located bulky switches or levers that tend to collect bacteria within crevasses and on the surfaces thereof. Moreover, some external switches and levers need to be constantly held by an individual to allow airflow and saliva suction, which can cause hand/arm fatigue over extended periods of time.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIG. 1 illustrates a saliva ejector suction system in accordance with an example of the present disclosure;

FIG. 2A illustrates a cross-sectional view of a saliva ejector device in a closed position in accordance with an example of the present disclosure;

FIG. 2B illustrates a cross-sectional view of the saliva ejector device of FIG. 2A in an open position;

FIG. 2C illustrates a fastening mechanism for the saliva ejector device of FIG. 2A in accordance with an example of the present disclosure;

FIG. 3A illustrates a cross-sectional view of a saliva ejector device in a closed position in accordance with an example of the present disclosure;

FIG. 3B illustrates a cross-sectional view of the saliva ejector device of FIG. 3A in an open position;

FIG. 3C illustrates a cross-sectional view of a saliva ejector device in an open position in accordance with an example of the present disclosure;

FIG. 4A illustrates a cross-sectional view of a saliva ejector device in a closed position in accordance with an example of the present disclosure;

FIG. 4B illustrates a cross-sectional view of the saliva ejector device of FIG. 4A in an open position;

FIG. 5A illustrates a cross-sectional view of a saliva ejector device in accordance with an example of the present disclosure; and

FIG. 5B illustrates a perspective view of a handle adapter in accordance with an example of the present disclosure.

Reference will now be made to the exemplary embodiments illustrated, and specific language will be used herein to describe the same. It will nevertheless be understood that no limitation of the scope of the invention is thereby intended.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

An initial overview of technology embodiments is provided below and then specific technology embodiments are described in further detail later. This initial summary is intended to aid readers in understanding the technology more quickly but is not intended to identify key features or essential features of the technology nor is it intended to limit the scope of the claimed subject matter.

In one example, disclosed is a saliva ejector device comprising a housing having a top opening and a bottom opening and a passageway extending between the top and bottom openings; an automatic switching mechanism supported in the housing, the automatic switching mechanism comprising a hollow compliant tube positioned in the passageway and supported about an interior portion of the housing; and a pressure-sensing operating switch disposed within a wall of the housing, and movable between a closed position and an open position upon application and release of pressure, respectively, thereon. In the closed position, the pressure-sensing operating switch compresses the hollow compliant tube to terminate airflow through the housing, and in the open position, the pressure-sensing operating switch facilitates the expansion of the hollow compliant tube to initiate airflow through the housing.

In another example, disclosed is a saliva ejector device comprising a housing having a top opening and a bottom opening and a passageway extending between the top and bottom openings; an automatic switching mechanism supported in the housing, the automatic switching mechanism comprising a pressure-sensing operating switch disposed within a wall of the housing, and movable between a closed position and an open position upon application and release of pressure, respectively, thereon, the pressure-sensing operating switch comprising a first end and a second end having a gate portion; and a compliant device supported between the housing and the pressure-sensing operating switch to bias the pressure-sensing operating switch in an open position. In the closed position, the gate portion engages the housing, such that the pressure-sensing operating switch terminates airflow through the housing, and in the open position, the pressure-sensing operating switch is disengaged from the housing to facilitate airflow through the housing.

In another example, disclosed is a saliva ejector device comprising a handle operable with a vacuum device and a saliva ejector conduit, and configured to be supported by a cradle, the handle comprising an upper housing having an upper passageway formed therein to facilitate airflow through the upper housing; and a lower housing having a lower passageway formed therein to facilitate airflow through the lower housing. The upper housing is rotatably coupled to the lower housing to selectively switch the handle between an open position with the upper passageway in fluid communication with the lower passageway to facilitates airflow through the handle, and a closed position with the upper passageway out of fluid communication with the lower passageway to restrict airflow through the handle.

In another example, disclosed is a saliva ejector adapter operable for use with a handle of a saliva ejector device operating within a saliva ejector system, the saliva ejector adapter comprising a handle adapter comprising a body section having an ergonomic configuration for facilitating grasping by a user with the hand in a functional position; a saliva ejector conduit interconnect formed about an upper opening of the handle adapter, the saliva ejector conduit interconnect having a configuration that facilitates coupling of the handle adapter to a saliva ejector conduit; a handle interconnect formed about a lower opening of the handle adapter, the handle interconnect having a configuration that facilitates coupling of the handle adapter to a handle of a saliva ejector device; and a passageway extending through the handle adapter and in fluid communication with the upper opening and the lower opening.

In one example there is provided a saliva ejector device comprising a housing or handle having a passageway between a top opening and a bottom opening of the handle. The bottom opening is coupleable to a vacuum device operable to vacuum body fluids through the passageway. The handle sized and shaped to be vertically supported by a cradle. The saliva ejector device can comprise an automatic switching mechanism coupled to the handle having a pressure-sensing operating switch or button disposed through an exterior wall of the handle. The automatic switching mechanism is designed to be switchable between an open position (that facilitates airflow through the passageway) and a closed position (that restricts airflow about the passageway). The button can be designed to be automatically depressed by the cradle when the handle is placed in the cradle, thereby switching to the closed position. The button can be designed to be automatically released when the handle is removed from the cradle, thereby switching to the open position. A compliant device, such as a compliant tube or a spring, can be positioned in the passageway to assist with automatic release of the button when switching to the open position.

In one example there is provided a method of facilitating automatic switching of airflow about a saliva ejector device. The method can comprise providing a saliva ejector device supported in a cradle. The saliva ejector device comprises a handle having a passageway selectively in fluid communication with a vacuum device and a saliva ejector conduit. The method can comprise removing the saliva ejector device from the cradle, thereby automatically releasing a button of the handle by the cradle, and thereby allowing airflow about the passageway of the handle. The method can comprise placing the saliva ejector device into the cradle, thereby automatically depressing the button with the cradle to restrict airflow about the passageway of the handle.

In one example there is provided a saliva ejector device comprising a housing or handle coupleable to and operable with a vacuum device and a saliva ejector conduit, and designed and configured to be supported (e.g., vertically) by a cradle. The handle can comprise an upper housing having an upper passageway formed therein to facilitate airflow through the upper housing, and a lower housing having a lower passageway formed therein to facilitate airflow through the lower housing. The upper housing is rotatably coupled to the lower housing to selectively switch the handle between an open position (that facilitates airflow through the handle) and a closed position (that restricts airflow about the handle). The upper passageway is designed to be in fluid communication with the lower passageway when in the open position, and the upper passageway is designed to not be in (or out of) fluid communication with the lower passageway when in the closed position.

In one example there is provided a method of making a rotatable saliva ejector device. The method can comprise forming an upper housing and an upper passageway through the upper housing, whereby the upper housing is coupleable to a saliva ejector conduit. The method can comprise forming a lower housing and a lower passageway through the upper housing, whereby the lower housing is coupleable to a vacuum conduit. The method can comprise rotatably coupling the upper housing to the lower housing wherein the upper housing rotates relative to the lower housing to selectively allow or restrict airflow about the handle.

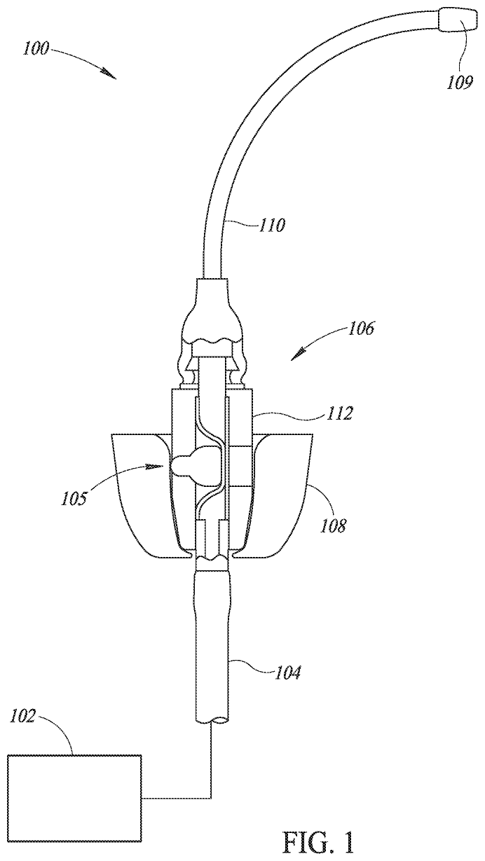

FIG. 1 illustrates a perspective view of an exemplary saliva ejector suction system 100 in accordance with one example. The saliva ejector suction system 100 can comprise a vacuum device or source 102 configured to provide a source for supplied vacuum suction, a vacuum conduit 104, a saliva ejector device 106, a cradle 108, and a saliva ejector conduit 110. The vacuum conduit 104 can be fluidly coupled to the vacuum device 102, and can be removably and fluidly coupled to the saliva ejector device 106 to facilitate vacuum suction by the saliva ejector device 106 as is known in the art. The saliva ejector conduit 110 can be removably coupled (selectively attached and detached) to the saliva ejector device 106. In operation, a dental practitioner or other individual can operate the saliva ejector suction system 100 by turning on the vacuum device 102 to initiate airflow and vacuum suction. The saliva ejector conduit 110, having a saliva ejector tip 109, can be inserted into the mouth of the patient with air flowing (e.g., suction flow) through the saliva ejector conduit 110, the saliva ejector device 106, and through the vacuum conduit 104 for disposal in the vacuum device or other waste system.

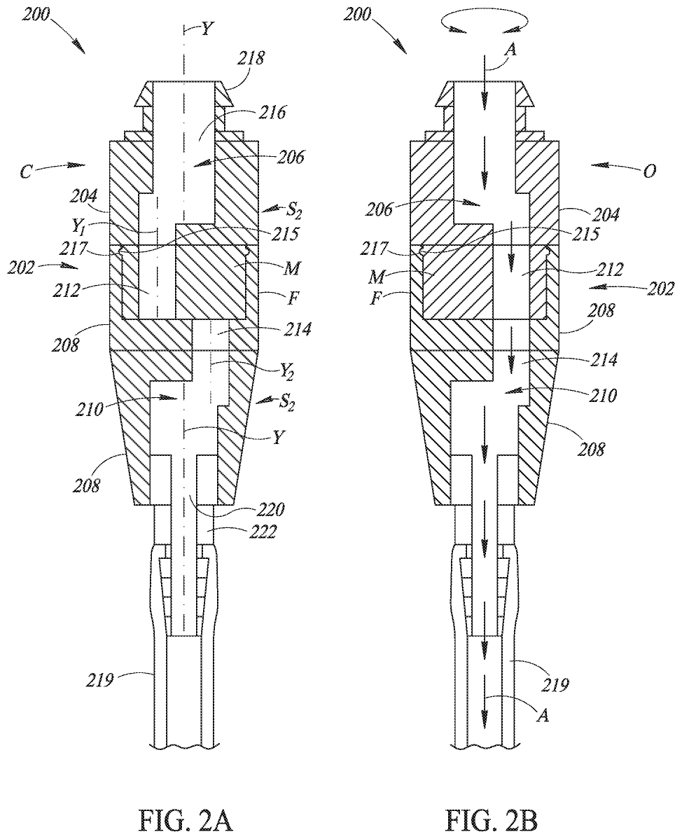

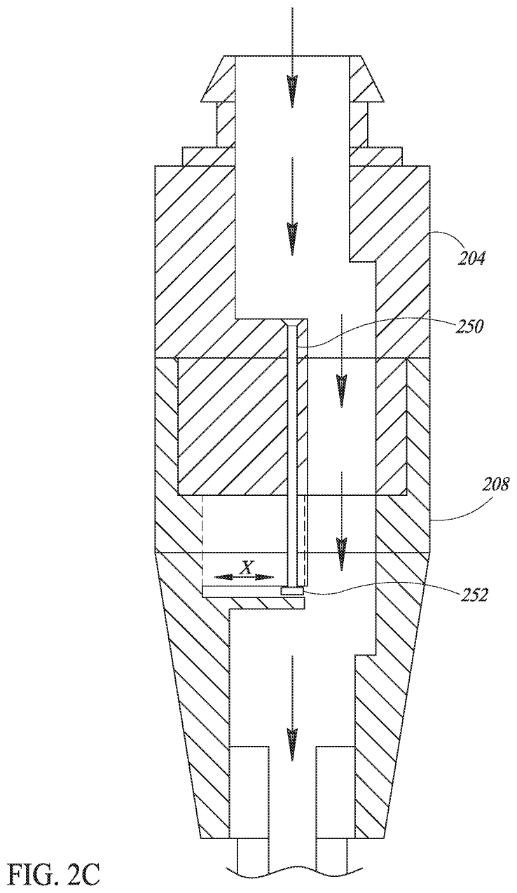

The saliva ejector device 106 can comprise a housing or handle 112 that can be removable from and vertically supported by a holder or cradle 108. The cradle 108 can be a known cradle used in the industry that can temporarily hold or cradle the saliva ejector device 106, such as when not in use by a user. The cradle 108 can be fastened to a stand (not shown) or other support structure. While in the cradle 108, airflow about the handle 106 can be restricted or blocked by a switching mechanism 105, such as the switching mechanisms described below with reference to FIGS. 2A-5A. Therefore, when the saliva ejector device 106 is in the cradle 108, the handle 112 can be in a closed position, as further discussed herein. The saliva ejector device 106 and the cradle 108, each of the saliva ejector suction system 100, can each be configured to operate with one another, such that insertion of the saliva ejector device 106 into the cradle 108 can cause a stop in the flow of air and a cessation of the suction operation. Likewise, removal of the saliva ejector device 106 from the cradle 108 can cause an initiation of the flow of air and a continuation of the suction operation. As will be further exemplified below, the switching mechanism 105 and the handle 110 can be configured so as to collectively form a substantially uniform exterior surface of the handle 110 of the saliva ejector device 106. The saliva ejector conduit 110 can be removably coupled to any of the saliva ejector device examples discussed below.

FIGS. 2A and 2B show one example of a saliva ejector device 200 in a closed position C (FIG. 2A) and an open position O (FIG. 2B). The saliva ejector device 200 comprises a housing or handle 202 comprising an upper housing 204 having an upper passageway 206 and a lower housing 208 comprising a lower passageway 210. The upper housing 204 is rotatably coupled to the lower housing 208 to selectively switch the device 200 (and the handle 202) between the open position O (that facilitates airflow A through the handle 202) and the closed position C (that restricts or blocks airflow A through the handle 202). In practice, an individual can manually rotate the housings 204 and 208 relative to each other to effectively turn the saliva ejector device 200 (and system) on and off.

In the open position O the upper passageway 206 is in fluid communication with the lower passageway 210. And, in the closed position C the upper passageway 206 is not in or is out of fluid communication with the lower passageway 210. More specifically, the handle 202 has a central axis Y that extends centrally along the handle 202 from top to bottom, as shown. The upper housing 204 has an upper passageway 206 comprising a central passageway portion and an offset passageway portion 212. The offset passageway portion 212 is formed to have a central axis Y₁ that is offset relative to the central passageway portion of the upper passageway 206 formed along the central axis Y of the handle 202. Indeed, the upper housing 204 is structurally configured such that the offset passageway portion 212 formed therein is situated along the Y₁ axis offset from the central passageway portion of the upper passageway 206 formed along the central axis Y. The offset and central passageway portions make up the upper passageway 206, and are configured to define a fluid (e.g., air) pathway through the upper housing 204. Likewise, the lower housing 208 has a lower passageway 210 comprising a central passageway portion and an offset passageway portion 214. The offset passageway portion 214 is formed to have a central axis Y₂ that is offset relative to the central passageway portion of the lower passageway 210 formed along the central axis Y of the handle 202. Indeed, the lower housing 208 is structurally configured such that the offset passageway portion 214 formed therein is situated along the Y₂ axis offset from the central passageway portion of the lower passageway 210 formed along the central axis Y. The offset and central passageway portions make up the lower passageway 210, and are configured to define a fluid (e.g., air) pathway through the lower housing 208.

In the closed position C of FIG. 2A, the upper housing 204 and the lower housing 208 are rotated relative to one another, such that the offset passageway 212 (of the upper housing 204) and its central axis Y₁ is spatially and structurally separated from the offset passageway 214 (of the lower housing 208) and its central axis Y₂. Therefore, the upper passageway 206 is ‘closed-off’ from fluid communication with the lower passageway 210. Conversely, in the open position O of FIG. 2B, the upper housing 204 and the lower housing 208 are rotated relative to one another, such that the offset passageway 212 and its central axis Y₁ is spatially and structurally aligned with the offset passageway 214 and central axis Y₂. In this position, the upper passageway 206 is in fluid communication with the lower passageway 210, as shown, as a result of the alignment of the offset passageway portions 212 and 214 of the upper and lower housings 204 and 208, respectively.

The upper and lower housings 204 and 208 can be rotatably coupled to one another in a variety of ways. In one example, the lower housing 208 can be configured with a female end or portion having a female interface portion F to rotatably receive a male end or portion of the upper housing 204 having an interface portion M. The female end and interface portion F can be a bore formed through an end of the lower housing 208, and the male end and interface portion M can be a protrusion formed at an end of the upper housing 204, as shown. These interface portions can be designed and configured to rotate about each other along the Y axis, as illustrated between the views of FIGS. 2A and 2B.

The housings 204 and 208 can be rotatably secured to each other in a variety of ways. In one example, as illustrated in FIGS. 2A and 2B, the upper housing 204 can be snap-fit or frictionally fit to the lower housing 208. For instance, the upper housing 204 can have a protrusion 215 that is slidably seated in a recess 217 of the lower housing, much like a tongue and groove interface. The protrusion 215 and the recess 217 can be formed linearly about respective circumference surfaces of a respective housings 204 and 208. Thus, the housings 204 and 208 can be coupled together by applying sufficient force to “snap” or otherwise engage the protrusion 215 into the recess 217, thereby preventing the housings from coming apart and while still allowing for the rotational movement of the housings 204 and 208 as discussed herein.

In another example, the housings 204, 208 can be coupled together using a fastener, as illustrated in FIG. 2C. In this example, a fastener 250 slidably couples the upper housing 204 to the lower housing 208. Specifically, the fastener 250 is secured through a hole in the upper housing 204 and extends through a hole in the lower housing 208. A nut 252 may be secured to an end of the fastener 250 to retain the fastener 250 to the housings 204 and 208. The nut 252 may be slidably received in a radial slot 254 formed through the lower housing 208, and a section of the fastener 250 may be slidable within a radial slot 255 formed through the lower housing 208 to correspond to the configuration of the slot 254. As illustrated by the direction of arrows X, the fastener 250 and the nut 252 are laterally (and radially) moveable within the lower housing 208 as the upper housing 204 is rotated relative to the lower housing 208, for example. Moreover, the handle 202 can comprise a seal or sealing means operable to seal the upper and lower housings 204, 208, thus preventing blood, bacteria, and/or foreign matter or other contaminants from getting into the interior of the handle 202. It is noted that those skilled in the art will recognize still other ways to slideably or rotatably couple together the housings 204 and 208, and as such, the specific examples discussed above and shown in the drawings are not intended to be limiting in any way.

The housings 204 and 208 can each be at least partially cylindrically shaped. In one example the upper portion or area of the lower housing 208 can be cylindrical in shape, with a lower portion or area of the lower housing 208 extending therefrom, and being conically tapered, as shown. The lower housing 208, or at least a portion thereof, can be received loosely by a cradle (e.g., see FIG. 1). As shown, but not intending to be limiting in any way, the upper housing 204 can be substantially cylindrical in shape. Together, the housings 204 and 208 form a handle that has a substantially uniform exterior surface S. This means that most or the entire exterior surface of the handle 202 is substantially uniform along its cylindrical and tapered conical surfaces about which an individual holds the handle 202. In other words, there are no protrusions extending from the uniform exterior surface of the housings 204 and 208 (protrusions or other devices such as switches, buttons, or other features extending therefrom). This improves or maximizes desired sanitation conditions compared to handles that have such protrusions because bacteria has less surfaces and crevasses to collect therein or thereon. This also improves ergonomics of the handle 202 as there are no external switches, etc, that can interfere with a user's grip of the handle 202, or in other cases as an individual is not required to hold a button or other switch the entire time when operating the saliva ejection device 200.

The upper housing 204 further comprises a top opening 216 formed through a saliva ejector conduit interconnect (e.g., nipple 218) configured to receive and couple a saliva ejector conduit (e.g., FIG. 1). Likewise, the lower housing 208 comprises a bottom opening 220 formed through a vacuum conduit interconnect (e.g., nipple 222) configured to receive and couple a vacuum conduit 219. Although not specifically shown, it is to be understood that the saliva ejector device 200 can be operable with a saliva ejector conduit, such as the saliva ejector conduit 110 discussed above, as well as with a vacuum device, such as the vacuum device 102 discussed above, the saliva ejector device 200 being operable within a saliva ejector suction system.

A method of making the saliva ejector device 200 can comprise forming the upper housing and an upper passageway through the upper housing and forming the lower housing and a lower passageway through the lower housing. These upper and lower housings (e.g., 204 and 208) can be formed of metal, such as stainless steel, and machined to have and define said passageways there through. Alternatively, the housings can be molded of a polymer, plastic, or composite material. The upper and lower housings (e.g., 204 and 208) can be rotatably coupled to each other (e.g., circumferentially rotated, wherein the upper and lower housings 204, 208 rotate about a common axis, such as axis Y) such that the upper housing rotates relative to the lower housing to selectively allow or restrict airflow about the handle, as further described above. The aforementioned male and female end portions and corresponding interfaces can be machined or otherwise formed as shown on FIG. 2A to facilitate said rotatable motion.

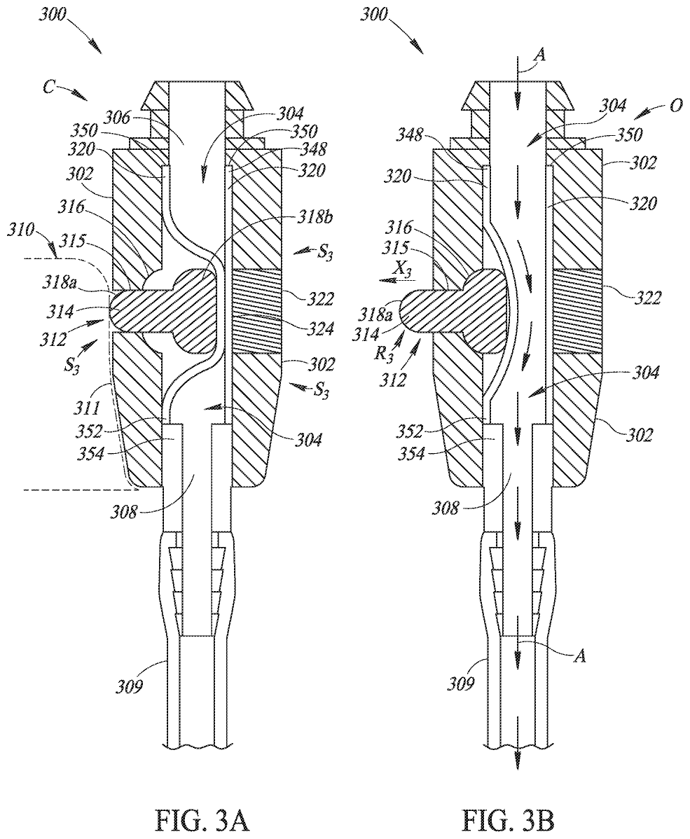

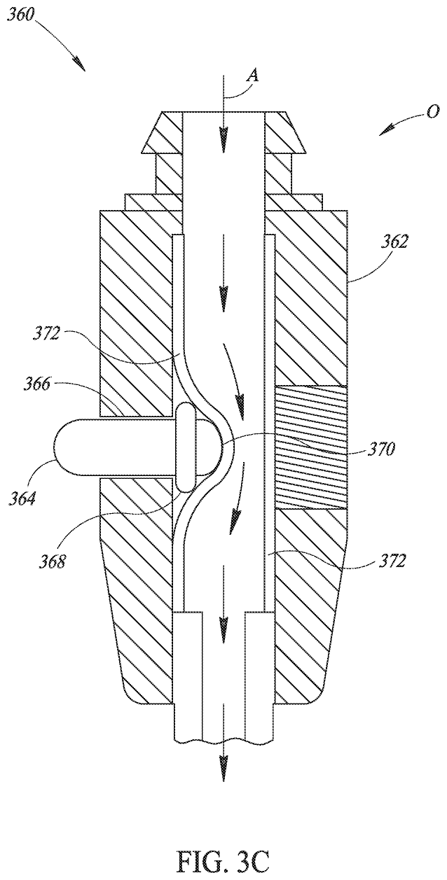

FIGS. 3A and 3B show an example of a saliva ejector device 300 in accordance with another example. The saliva ejector device 300 can comprise a closed position and configuration C (FIG. 3A) and an open position and configuration O (FIG. 3B). The saliva ejector device 300 comprises a housing (e.g., in the form of a handle 302, or at least the handle 302 comprising at least a portion of the housing) and a passageway 304 that extends between a top opening 306 and a bottom opening 308 of the handle 302. The handle 302 can comprise a vacuum conduit interconnect that can couple to a vacuum conduit 309 of a vacuum device (not shown, but see FIG. 1 example), such that the bottom opening 308 is in fluid communication with the vacuum conduit 309 of the vacuum device that suctions body fluids and debris through the passageway 304. Likewise, the handle 302 can comprise a saliva ejector conduit interconnect (e.g., see nipple portion of the handle 302 formed about the top opening 306) that can couple to a saliva ejector conduit (not shown, but see example in FIG. 1), such that the top opening 306 is in fluid communication with the saliva ejector conduit. Although not specifically shown, it is to be understood that the saliva ejector device 300 can be operable with a saliva ejector conduit, such as the saliva ejector conduit 110 discussed above, as well as with a vacuum device, such as the vacuum device 102 discussed above, the saliva ejector device 300 being operable within a saliva ejector suction system.

As shown in FIG. 3A, the handle 302 is sized and shaped to be supported (e.g., vertically) by a cradle 310. In one example, the handle 302 can comprise a tapering cylindrical lower portion that corresponds in size and shape (only slightly smaller) to a tapering cylindrical cradle having an interfacing surface that engages the handle 302 upon inserting the handle 302 therein. FIG. 3A only shows a left interfacing surface 311 of the cradle 310 in dashed lines (however, see FIG. 1 showing an entire example cradle).

The saliva ejector 300 further comprises an automatic switching mechanism 312, which comprises a pressure-sensing operating switch or button 314 supported within a wall of the handle 302 and disposed through an aperture 315 formed in/through the wall 316 of the handle 302. As shown, although not intending to be limiting in any way, the button 314 comprises a first end 318a and a second end 318b, with the first end 318a being formed as a nipple or protrusion extending from the second end 318b, the second end 318b being shaped similar to a head of a hammer and comprising a larger cross-sectional area or width (taken along a longitudinal axis of the handle 302) than the first end 318a. The housing 302 can further comprise a cutout extending from the aperture 315, the cutout defining surfaces having a similar shape and contour as that of the second end 318b of the button 314. As so configured, the nipple configuration of the first end 318a can extend bi-directionally in and out of the opening or aperture 315, and in the fully outward and extended position shown in FIG. 3B, the second end 318b can be seated about the corresponding surfaces of the cutout of the handle 302 adjacent the aperture 315 of the exterior wall 316 (i.e., much like a male-female interface). The aperture 315 can be formed through the handle 302 a particular or sufficient distance such that the handle 302 (via the aperture 315) structurally and slidably supports the first end 318a of the button 314 when it is depressed to prevent the button 314 from coming loose or failing to properly operate (as similarly illustrated on FIG. 3C, showing a horizontal aperture receiving a horizontal button). In one aspect, the button 31 can be supported directly about and can slide directly about the walls of the handle 302 defining the aperture 315 formed in the handle 302. In another aspect, a bushing or other interface device can be supported by the walls of the handle 302 about the aperture 315, wherein the bushing or other interface device is caused to engage the surfaces of the button 314 in order to provide a more secure coupling of the button 314 to the handle 302, and also, depending upon the material of the bushing or other interface device, to provide a reduced friction interface between the button 314 and the handle 302.

The switching mechanism 312 can further comprise a hollow compliant tube 320 positioned or disposed vertically within the passageway 304 and supported about an interior of the handle 302, such as extending from the top opening 306 to the bottom opening 308. Given application of a sufficient pressure or force, the compliant tube 320 is selectively compressible to close off and open (or block and permit) airflow through the passageway 304. In the example shown, the button 314 can be supported within the handle 302, and can be movable in a bi-directional manner to act on the compliant tube 320. In one example (as shown in FIG. 3A), the button 314 can be actuated and caused to be moved sufficiently inward toward the compliant tube 320, such that the second end 318a of the button 314 is caused to act on the compliant tube 320 to collapse or pinch or compress the compliant tube 320 against the inner walls of the handle 302, thereby restricting and cutting off airflow through the passageway 304 of the handle 302. Conversely, the button 314 can be deactivated and caused to move outward away from the compliant tube 320, thereby facilitating the expansion of the compliant tube 320 in a direction to again open the passageway 304. In this manner, the switching mechanism 312 can be selectively actuated to switch the device 300 between the open position O and the closed position C, thereby allowing or restricting airflow (respectively) through the passageway 304 and the handle 302.

The button 314 can be automatically actuated between open and closed positions by either removing the device 300 from the cradle 310 or placing the device 300 into the cradle 310, the cradle being configured with one or more receiving surfaces sufficient to apply a suitable force to the button 314 as the handle 302 is inserted into the cradle 310. Stated differently, as the handle 302 is inserted into a suitably configured cradle having a slightly larger cross-sectional diameter than the handle 302, the receiving surfaces on the cradle 310 can act on the button 314 to cause the button to move inward (depress the button) to overcome the biasing forces applied by the compliant tube 320 on the button 314 and to collapse the compliant tube 320. In this sense, the pressure sensing operation and the termination of airflow is automatic in that insertion of the saliva ejector device into the cradle automatically terminates the airflow without the user having to manually depress or manipulate the button 314 as the receiving surface of the cradle applies pressure to the button 314 sufficient to depress and activate the button 314 upon the saliva ejector device being inserted therein. Likewise, removal of the saliva ejector device from the cradle housing causes the button 314 to move or displace away from the tubing as the force from the cradle housing is removed.

In another aspect, if the saliva ejector device 300 is out of the cradle 310 already, such as during use, a user can selectively activate and deactivate the button 314 by manually). As specifically shown, when in the closed position C of FIG. 3A, the first end 318a of the button 314 is pressed inwardly toward the compliant tube 320 by lateral pressure exerted on the button 314 by the interfacing surface 311 of the cradle 310 (i.e., the gravitational or user applied forces imparted on the mass of the handle (and the conduits) transfers energy to cause the lateral pressure force to the button 314 via the cradle 310). This causes the second end 318b of the button 314 to press and pinch the compliant tube 320 against an inner wall 324 of the handle 302, which pinches the walls of the compliant tube 320 against themselves, thereby restricting or eliminating airflow A through the passageway 304 (FIG. 3A).

The button 314 can comprise a radial surface configuration R₃ that facilitates application of an axial inward force to the button 314 as the button 314 interfaces with the surface 311 of the cradle 310, namely when the handle 302 is placed into the cradle 310. The radial surface configuration R₃ can be configured to receive a direct force in a direction transverse to the direction of movement of the button 314, wherein a resulting or component force vector that is in the direction of movement of the button 314 can be great enough to displace the button 314 inward, collapse the compliant tube 320, and close off the passageway 304. The radial surface configuration can also function to reduce the amount of surface area of the button 314 in contact with the cradle. Thus, the button 314 can tend to slide more easily about the cradle 310 when an individual places or removes the handle 302 within/from the cradle 310.

The compliant tube 320 can be configured to comprise a degree of potential energy when collapsed, the potential energy being sufficient to expand the compliant tube 320 and act on and bias the button 314 to move it in an outward direction (such as when the device 300 is out of the cradle and no counter forces are acting on the button 314), thus opening airflow through the tube 320 and the passageway 304. Accordingly, as illustrated in FIG. 3B, when the handle 302 is removed from its cradle 310 by an individual, the compliant tube 320 releases its potential energy to return to its normal (open) position, thereby applying a biasing force to and actuating the button 314 outwardly, as shown by arrow X₃. Thus, the compliant tube 320 opens a path to allow airflow A through the tube 320 within the passageway 304. Accordingly, the compliant tube 320 acts like a spring and a conduit at the same time.

The compliant tube 320 can be comprised of a plastic material that is flexible enough to be pinched by the force of the button (e.g., about 1-5 pounds of force), yet compliant enough to “spring-back” to its normal position when actuating the button 314 outwardly through the aperture 315 of the handle 302. A plug 322 can be disposed through a right bore in the handle 302, which seals the bore that is used to install the tube 320 and the button 314.

The compliant tube 320 can be coupled to or supported by the handle 302 in a variety of ways. For example, the compliant tube 320 can have an upper end 348 that is seated or abutted against flange 350 formed circumferentially into the handle 302. Thus, the flange 350 could be defined by a diameter change of the inner surface of the handle 302 making up the passageway 304. Similarly, a lower end 352 of the compliant tube 320 could be seated or abutted against an end of a coupling member 354 that extends into the lower opening 308 of the handle 302. In one example, either end (and/or outer surfaces) of the compliant tube 320 could be adhered (e.g., with an adhesive) or otherwise attached (e.g., with one or more fasteners or couplers) to the handle 302 and/or the coupling member 354, which could still allow the compliant tube 320 to be manipulated between closed and open positions. In some examples, the compliant tube can be part of the vacuum conduit or the saliva ejector conduit, meaning that a compliant tube can extend from one of the conduits into and through the handle.

As illustrated in FIG. 3A, the switching mechanism 312 (i.e., button 314 and tube 320) and the handle 110 collectively form a substantially uniform exterior surface S3 of the handle 302. This means that most of the entire exterior surface of the handle is substantially uniform along its cylindrical and tapered conical surfaces about which an individual holds the handle. In other words, when the handle 302 is placed into the cradle 310, there are no protrusions extending from the uniform exterior surface of the handle (protrusions or other devices such as switches, buttons, or other features extending therefrom). And, when the handle 302 is removed from the cradle 310, there is only a single, small smooth surface device (e.g., the button 314) extending from the handle 302. Both instances tend to improve or maximize desired sanitation conditions compared to handles that have such protrusions because bacteria has less surfaces and crevasses to collect therein. This also improves ergonomics of the handle because an individual is not required to hold a button or other switch the entire time when operating the handle to suction body fluids.

FIG. 3C illustrates an alternative saliva ejector device 360 having a handle 362. The saliva ejector device 360 can have many of the structure and operation functions as described with reference to FIGS. 3A and 3B, such as a pressure-sensing operating switch or button that depresses a compliant tube to restrict airflow, and such structure and operation functions are intended to be incorporated here, as applicable. Some notable differences, however, are that the saliva ejector device 360 comprises a button 364 that has an elongated body that is slidably received through an aperture 366 of the handle 362. The button 364 (or actuation member) can comprise a flange 368 that acts as a stop member against an inner wall of the handle 362 to limit the range of motion of the button 364, and to prevent the button 364 from passing all the way through the handle 362. The button 364 can have an interfacing surface 370 that engages a compliant tube 372, such that the button 364 is held in a biased or loaded state by the compliant tube 372. Stated differently, the compliant tube 372 applies a continuous outward force on the button 364 in a direction to cause the button to move outward and away from the compliant tube 372. When the button 362 is depressed inwardly, the forces applied by the compliant tube 372 are overcome and airflow through the passageway is at least partially blocked or fully blocked depending upon the displacement distance of the button 364, similarly as described with reference to the movement and function of the second end 318b of button 314 of FIGS. 3A and 3B.

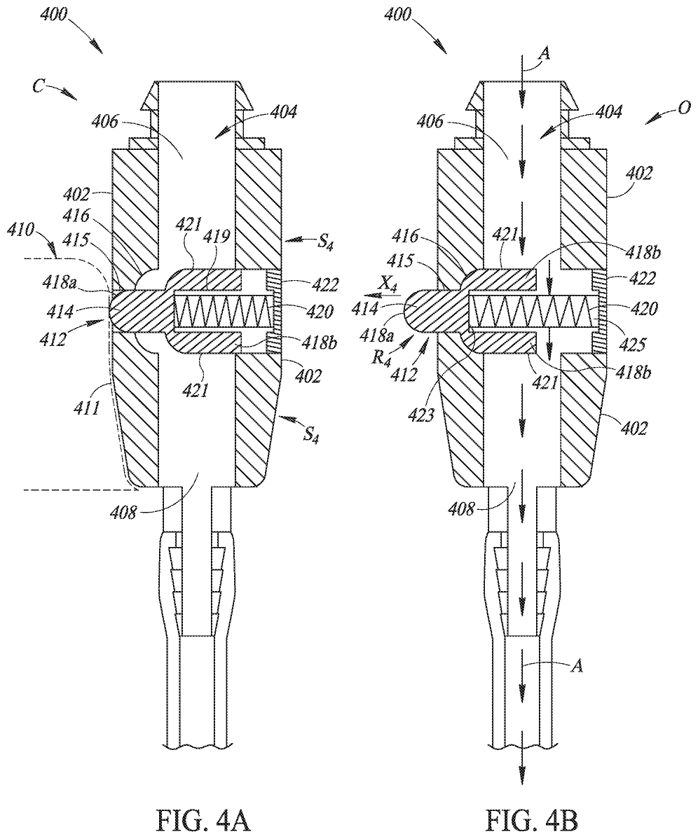

FIGS. 4A and 4B show another example of a saliva ejector device 400. The saliva ejector device 400 can comprise a closed position and configuration C (FIG. 4A) and an open position and configuration O (FIG. 4B). As best shown on FIG. 4B, the saliva ejector device 400 comprises a housing (e.g., in the form of a handle 402, or at least the handle 402 comprising at least a portion of the housing) and a passageway 404 that extends between a top opening 406 and a bottom opening 408 of the handle 402. The handle 402 can comprise a vacuum conduit interconnect that can couple to a vacuum conduit 409 of a vacuum device (not shown, but see FIG. 1 example), such that the bottom opening 408 is in fluid communication with the vacuum conduit of the vacuum device that suctions body fluids and debris through the passageway 404. Likewise, the handle 402 can comprise a saliva ejector conduit interconnect (e.g., see nipple portion of the handle 402 formed about the top opening 406) that can couple to a saliva ejector conduit (not shown, but see example in FIG. 1), such that the top opening 406 is in fluid communication with the saliva ejector conduit. Although not specifically shown, it is to be understood that the saliva ejector device 400 can be operable with a saliva ejector conduit, such as the saliva ejector conduit 110 discussed above, as well as with a vacuum device, such as the vacuum device 102 discussed above, the saliva ejector device 400 being operable within a saliva ejector suction system.

As shown in FIG. 4A, the handle 402 is sized and shaped to be supported (e.g., vertically supported) by a cradle 410. In one example, the handle 402 can comprise a tapering cylindrical lower portion that corresponds in size and shape (only slightly smaller) to a tapering cylindrical cradle having an interfacing surface that engages the handle 402 upon inserting the handle 402 therein. FIG. 4A only shows a left interfacing surface 411 of the cradle 410 in dashed lines (however, see FIG. 1 showing an entire example cradle).

The saliva ejector device 400 further comprises an automatic switching mechanism 412, which can comprise a pressure-sensing operating switch or button 414 disposed through an aperture 415 formed through an exterior wall 416 of the handle 402. As shown, although not intending to be limiting in any way, the button 414 comprises a first end 418a and a second end 418b, the button 414 being movable in a bi-directional manner relative to the handle 402. The first end 418a can be formed as a nipple or protrusion extending from the second end 418b. The handle 402 can further comprise a cutout extending from the aperture 415, the cutout defining surfaces having a similar shape and contour as that of the second end 418b of the button 414. As so configured, the nipple configuration of the first end 418a can extend bi-directionally in and out of the opening or aperture 415, and in the fully outward and extended position shown in FIG. 4B, the second end 418b can be seated about the corresponding surfaces of the cutout of the handle 402 adjacent the aperture 415 of the exterior wall 416 (i.e., much like a male-female interface). The aperture 415 can be formed through the handle 402 a particular or sufficient distance such that the handle 402 (via the aperture 415) structurally and slidably supports the first end 418a of the button 414 when it is depressed to prevent the button 414 from coming loose or failing to properly operate.

The second end 418b can comprise a gate portion 421 configured to move laterally and to be selectively positioned within the passageway 404 to facilitate the passage and blocking or occluding of airflow through the passageway 404. The gate portion 421 can have a bore 419 formed laterally therein. In another aspect, the first end 418a of the button 414 can be a separate component from the gate portion 421, and can be attached to each other by a weld, adhesive, or other suitable attachments. Upon application of pressure to the button 414, the gate portion 421 can be moved and positioned to engage the housing (e.g., an inner wall defining the passageway 404), thereby blocking off airflow through the handle 402 and the housing. As shown in FIG. 4A, the gate portion 421 is positioned in the closed position, such that it is seated within a recess of the inner wall of the housing, thus engaging the housing to seal off the passageway 404. The gate portion 421 and the first end of the button 414 thereby operate to block or terminate airflow through the passageway 404. Upon release of the pressure or force to the button 414, button 414 moves to an open position facilitating airflow through the passageway 404.

The switching mechanism 412 can further comprise a compliant or biasing device 420 (an energy storage device), such as a spring, operatively situated and supported between the button 414 and the handle 402 in order to apply a biasing force to the button 414 in an outward direction relative to the handle 402 (to the left in the view shown), to place the button 414 in the open position with the gate portion 421 disengaged from the housing to permit airflow through the passageway. In one example, the spring can be situated and supported within the bore 419 of the second end 418b of the button 414, the spring extending between an interface portion 423 of the button 414 and an interface portion 425 of the handle 402 (FIG. 4B), and the spring configured to apply a biasing force to the button 414 in an outward direction to open the passageway 404. Therefore, the compliant device 420 can be at least partially disposed through the bore 419 of the button 414. Those skilled in the art will recognize that in other examples, the spring may be supported in a different configuration to apply the biasing force to the button 414, the specific example shown herein not intending to be limiting in any way. In addition, those skilled in the art will recognize that the biasing or compliant device 420 can comprise a spring, a rubber member, or any other energy storage device/system.

The button 414 and corresponding gate portion 421 are actuated laterally about the passageway 404 when switching to the open position O, in a direction as shown by arrow X₄, by the force of the compliant device 420 expanding laterally. The force exerted by the compliant device 420 causes the button 414 and its gate portion 421 to move to the open position O (FIG. 4B). FIG. 4A shows the compliant device 420 in a compressed state while FIG. 4B shows the compliant device 420 in an expanded state (but the compliant device may still remain slightly compressed in FIG. 4B to ensure airflow around the gate portion).

Similar to the example device 300 of FIGS. 3A and 3B, the button 414 can be automatically actuated between the open and closed positions by either removing the device 400 from the cradle 410 or placing the device 400 into the cradle 410 (or when out of the cradle, the individual user can selectively activate the button manually). When in the closed position C of FIG. 4A, the first end 418a of the button 414 is pressed and moved inwardly toward the compliant device 420 by the interfacing surface 411 of the cradle 410 (i.e., the gravitational or user applied forces imparted on the mass of the handle (and the conduits) transfers energy to cause the lateral pressure force to the button 414 via the cradle 410). This causes the gate portion 421 to also move inward. The button 414 is thus moveable in an inward direction to overcome the forces of the compliant device 420. The button 414 is thus positioned to close about the passageway 404 with the compliant device 420 compressed, thereby restricting or eliminating airflow A through the passageway 404. Conversely, with the button positioned to close the passageway 404, removing the external force applied to the button 414 (e.g., by the cradle 410) causes the compliant device 420 to release its stored potential energy and to apply a force to the button 414 in a direction to move the button 414 outward, whereby the gate portion 421 is caused to be moved to a positon to open the passageway 404.

Similar to the button 314 discussed above and shown in FIGS. 3A and 3B, the button 414 can comprise a radial surface configuration R₄ that facilitates application of an axial inward force to the button 414 as the button 414 interfaces with the interfacing surface 411 of the cradle 410, namely when the handle 402 is placed into the cradle 410. A plug 422 can be disposed through a right bore in the handle 402, which seals the bore used to install the button 414 and compliant device 420.

As illustrated in FIG. 4A, the switching mechanism 412 (i.e., button 314 and tube 320) and the handle 402 collectively form a substantially uniform exterior surface S₄ of the handle 402 of the saliva ejector device 400. This means that most of the entire exterior surface of the handle is substantially uniform along its cylindrical and tapered conical surfaces about which an individual holds the handle. In other words, when the handle 402 is placed into the cradle, there are no protrusions extending from the uniform exterior surface of the handle (protrusions or other devices such as switches, buttons, or other features extending therefrom). And, when the handle 402 is removed from the cradle 410, there is only a single, small smooth surface device (e.g., the button 414) extending from the handle 402. Both instances tend to improve or maximize desired sanitation conditions compared to handles that have such protrusions because bacteria has less surfaces and crevasses to collect therein. This also improves ergonomics of the handle because an individual is not required to hold a button or other switch the entire time when operating the handle to suction body fluids.

In any example discussed in the present disclosure, a “pressure-sensing operating switch” or “button” can mean any device, actuator, component, element, plunger, member that is depressible to facilitate allowing or eliminating airflow about a passageway of a handle.

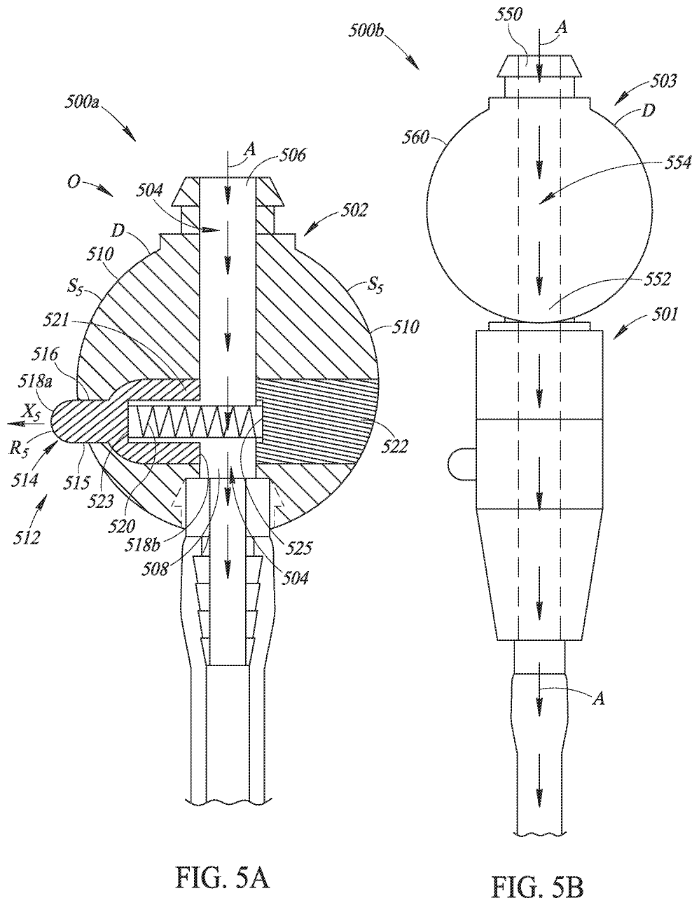

FIGS. 5A and 5B show different concepts of an ergonomic saliva ejector device 500a and 500b, respectively, operable with a saliva ejector system, such as that described above regarding FIG. 1. FIG. 5A shows the saliva ejector device 500a being configured with an automatic switching mechanism supported in the ergonomic handle, similarly as described in the examples above with reference to FIGS. 2A-4A. FIG. 5B shows the saliva ejector device 500b being configured with an ergonomic handle used as an adapter, or as a supplemental handle used in conjunction with an existing handle, such as handle 501 (which can comprise any of the handles of the saliva ejector device examples of FIGS. 2A-4B, or an existing conventional handle of a conventional saliva ejector device).

With reference to FIG. 5A, the saliva ejector device 500a has an ergonomic housing or handle portion 502 comprising a body section 510, which in this example is shown as being spherical. While holding a saliva ejector device (e,g., conventional devices, or those similar in size and configuration as discussed above, as well as handle 501 shown in FIG. 5B) for long periods of time, an individual can experience fatigue because of the relatively small cylindrical shape of the handle relative to most individual's hands (e.g., the hand begins to cramp over time while holding such handles). Therefore, the body section 510 is designed and shaped to be more ergonomic and to conform to an individual's hand (palm and finger(s)) to maximize or improve ergonomics of the handle 502 and reduce fatigue, as compared to the ergonomics provided by smaller handles, such as handle 501 shown in FIG. 5B, these having a smaller diameter than the diameter D of the spherically shaped handle 502. For instance, the diameter D of the spherical body section 510 can be between 1 and 3 inches as opposed to the diameter of less than an inch of the cylindrical handle 501 of FIG. 5B, which is similar in size to many conventional saliva ejector device handles. Moreover, the spherical body section 510 allows the user to effectively cup the handle with the fingers about the palm with the fingers in a more relaxed position. Indeed, having an increased handle size and ergonomic shape that conforms to the user's hand is ideal for holding over long periods of time because the hand and fingers are kept in more of the functional position rather than a flexed or extended position that can lead to fatigue and even long term adverse effects. Those skilled in the art will recognize that the body section 510 can comprise ergonomic shapes other than spherical. For example, it is contemplated herein that the body section 510 can comprise any shape designed to be more ergonomically correct and that places the hand in position that more closely resembles the functional position of the hand over prior conventional handle configurations, namely those that are cylindrical in nature. As such, a spherical body section is not intended to be limiting in any way.

The handle 502 comprises an upper opening 506 and a lower opening 508. The handle 502 can comprise a vacuum conduit interconnect configured to be coupled to a vacuum conduit of a vacuum device (not shown, but see FIG. 1 example), such that the bottom opening 508 is in fluid communication with the vacuum conduit, similar to the examples as discussed above. Likewise, the handle 502 can comprise a saliva ejector conduit interconnect (e.g., see nipple portion of the handle 502 formed about the top opening 506) that can couple to a saliva ejector conduit (see FIG. 1 example), such that the top opening 506 is in fluid communication with the saliva ejector conduit. Although not specifically shown, it is to be understood that the saliva ejector device 500a can be operable with a saliva ejector conduit, such as the saliva ejector conduit 110 discussed above, as well as with a vacuum device, such as the vacuum device 102 discussed above, the saliva ejector device 500a being operable within a saliva ejector suction system.

A passageway 504 extends in a vertical, central manner through the body section 510 of the handle 502, such that the passageway 504 places the upper opening 506 and the lower opening 508 in fluid communication with one another to facilitate airflow A through the handle 502. Those skilled in the art will recognize that the saliva ejector device 500a can be configured to comprise some or all of the features of the different examples described above with reference to FIGS. 2A-4B. For example, in one aspect, the saliva ejector device 500a can comprise a pair of rotatable housings (not shown), similar to the example of FIGS. 2A and 2B, to selectively turn on and off airflow. Alternatively, the saliva ejector device 500a can have a actuatable button biasing a compliant tube, similar to the example of FIGS. 3A and 3B, to selectively and automatically turn on and off airflow.

In one specific example, as shown in FIG. 5A, the saliva ejector device 500a can comprise an automatic switching mechanism 512 similar to the example of FIGS. 4A and 4B that comprises a pressure-sensing operating switch or button 514 disposed through an aperture 515 of an exterior wall 516 of the handle 502. The button 516 can comprise a first end 518a and a second end 518b as taught above. The second end 518b can comprise a gate portion 521 and a bore 519 formed therein, also as taught above. A compliant or biasing device 520, such as a spring, can be operatively situated and supported between the button 516 and the handle 502 in order to apply a biasing force to the button 516. The spring can be situated and supported within the bore 519, the spring extending between an interface portion 523 of the button 516 and an interface portion 525 of the handle 502 (i.e., the compliant device 520 is partially disposed through the bore 519), and the spring being configured to apply a biasing force to the button 516 in an outward direction to open the passageway 504, as taught above. The button 516 and corresponding gate portion 521 are actuated laterally through the passageway 504 when switching to the open position O, in a direction as shown by arrow X₅, by the force of the compliant device 520 expanding laterally. The force exerted by the compliant device 520 causes the button 516 and its gate portion 521 to move to the open position O, as shown in FIG. 5A. This allows airflow A, similarly as described regarding FIG. 4B.

The closed positon of the handle adapter 500a is not shown here, but it will be appreciated that the saliva ejector device 500a and the automatic switching mechanism 512 will facilitate an open position similar to that described above with reference to FIG. 4A. Essentially, when in the closed position, the button 514 is compressed inwardly toward the compliant device 520 by an interfacing surface of the cradle (e.g., FIG. 4A), which movement is sufficient to overcome the biasing force of the spring and to properly position the gate portion 521. This causes the gate portion 521 to close about the passageway 504, thereby restricting or eliminating airflow through the passageway 504.

The button 514 can comprise a radial surface R₅ similar to that described above. A plug 522 can be disposed through a bore in the handle 502, which seals the bore used to install the button 514 and the compliant device 520. The plug 522 can also serve as a seat for the compliant device 520, as shown.

A typical cradle known in the industry may need to be reshaped or redesigned to fit the spherically-shaped handle 502 for automatic actuation of the button 514. This can be achieved by molding or forming a cradle having a curved surface that corresponds to the curved surface of the handle 502 proximate the area around the button 514, such that a sufficient force is applied to the button 514 in order to actuate the button and overcome the bias on the button 514 by the spring (or other biasing element).

In another example, the saliva ejector device 500a can comprise an upper saliva ejector conduit interconnect about the upper or top opening 506, as discussed above, as well as a lower handle interconnect formed in the body section 510 of the handle 502 about the bottom opening 508 (as represented by the dotted lines), the upper and lower interconnects comprising the same configuration. In this example, the saliva ejector device 500a can be used as an additional or supplemental handle or an adapter operable with an existing saliva ejector device (not shown). Specifically, the handle 502 can be coupled about an existing saliva ejector device handle by coupling the lower handle interconnect (which can comprise an inverse size, shape and configuration as a saliva ejector conduit interconnect) to the existing saliva ejector device handle in the place of where a saliva ejector conduit would normally be coupled (e.g., coupled to the saliva ejector conduit interconnect of the existing handle). Once secured about the existing handle, a saliva ejector conduit can be coupled to the upper saliva ejector conduit interconnect of the handle 502. This concept is shown illustrated in FIG. 5B (although the saliva ejector conduit is not shown as being coupled to the handle adapter 503, but see FIG. 1 for an example of this). This concept is discussed in greater detail below.

With reference to FIG. 5B, shown is a saliva ejector device 500b in accordance with another example. In this example, the saliva ejector device 500b comprises an existing handle portion 501, which can be a conventional handle portion of a conventional saliva ejector device, or one in accordance with the examples discussed herein as pertaining to FIGS. 2A-4B.

The saliva ejector device 500b further comprises a saliva ejector adapter in the form of an ergonomic handle adapter 503, wherein the handle adapter 503 can be used as a supplemental handle adapted for use or operable with an existing handle (represented here by handle 501) of an existing saliva ejector device, or any of the example handles of the saliva ejector device examples of FIGS. 2A-4B to improve the ergonomics of the saliva ejector device. In the example shown, the handle adapter 503 can be configured with an ergonomic size and shape similar to the handle 502 and body section 510 of FIG. 5A, except in this example the saliva ejector device 500b does not comprise a switching mechanism, but is primarily an ergonomic adapter or add-on to be used with existing handles.

The handle adapter 503 can have an upper opening 550 and a lower opening 552 in fluid communication with a passageway 554 there between and extending through the handle adapter. The passageway 554 can be in fluid communication with the passageway of the handle 501. The handle adapter 503 can comprise a handle interconnect formed about the lower opening 552 having an interface configuration that mates with a saliva ejector conduit interconnect (not shown, but see example in FIG. 5A) of the handle 501, thus facilitating coupling of the handle adapter 503 to the handle 501 using the same type of connection that would otherwise be used to couple a saliva ejector conduit (e.g., see saliva ejector conduit 110 of FIG. 1) to the handle 501. Similarly, the handle adapter 503 can comprise a saliva ejector conduit interconnect formed about the upper opening 550 having an interface configuration that mates with a saliva ejector conduit (again, see saliva ejector conduit 110 of FIG. 1) to couple the saliva ejector conduit to the handle adapter 503. With the handle adapter 503 suitably coupled to the handle 501, the passageways of the saliva ejector conduit, the handle adapter 503, the handle 501, and the vacuum conduit (not shown, but see vacuum conduit 104 in FIG. 1) will be sufficiently aligned to permit the selective passage of airflow A there through. Moreover, the handle adapter 503 enhances the ergonomics of the overall saliva ejector device 500b by complementing the handle 501 and providing an additional (or substitute) handle and grasping portion to the user that allows the user to grip the saliva ejector device with the hand in a more functional position as compared to the position of the hand when only grasping the handle 501.

It is to be understood that the examples discussed are not limited to the particular structures, process steps, or materials disclosed herein, but are extended to equivalents thereof as would be recognized by those ordinarily skilled in the relevant arts. It should also be understood that terminology employed herein is used for the purpose of describing particular examples only and is not intended to be limiting.

Reference throughout this specification to “one example” or “an example” means that a particular feature, structure, or characteristic described in connection with the example is included in at least one embodiment of the present invention. Thus, appearances of the phrases “in one example” or “in an example” in various places throughout this specification are not necessarily all referring to the same example.

Furthermore, the described features, structures, or characteristics may be combined in any suitable manner in one or more examples. In the description, numerous specific details are provided, such as examples of lengths, widths, shapes, etc., to provide a thorough understanding of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A saliva ejector device operable with a vacuum device, the saliva ejector device comprising:
a handle configured to be supported by a cradle, the handle comprising:
a saliva ejector conduit interconnect configured to receive and couple a saliva ejector conduit, the saliva ejector conduit interconnect having an opening there through;
a vacuum conduit interconnect configured to receive and couple a vacuum conduit, the vacuum conduit interconnect having an opening there through;
a passageway operable to establish fluid communication between the opening of the saliva ejector conduit interconnect and the opening of the vacuum conduit interconnect;
a body section comprising an ergonomic outer surface extending away from the saliva ejector conduit interconnect and the vacuum conduit interconnect a direction and distance so as to facilitate placement of a user's hand in a functional position upon grasping the handle.

2. The saliva ejector device of claim 1, wherein the ergonomic outer surface of the body section of the handle comprises a spherical configuration.

3. The saliva ejector device of claim 1, wherein the housing comprises:
an upper housing having an upper passageway formed therein to facilitate airflow through the upper housing; and
a lower housing having a lower passageway formed therein to facilitate airflow through the lower housing,
wherein the upper housing is rotatably coupled to the lower housing to selectively switch the handle between an open position with the upper passageway in fluid communication with the lower passageway to facilitate airflow through the handle, and a closed position with the upper passageway out of fluid communication with the lower passageway to restrict airflow through the handle.

4. The saliva ejector device of claim 3, wherein the upper housing and the lower housing each comprise a hemispherical shape and define, at least in part, the body section of the housing, such that the ergonomic outer surface of the body section of the housing comprises a spherical shape.

5. The saliva ejector device of claim 3, wherein the upper housing is circumferentially rotatable about the lower housing.

6. The saliva ejector device of claim 3, wherein the upper passageway comprises an offset passageway portion positioned offset relative to a central axis of the handle, and wherein the lower passageway comprises an offset passageway portion positioned offset relative to the central axis of the handle.

7. The saliva ejector device of claim 6, wherein the offset passageway portion of the upper passageway is in fluid communication with the offset passageway portion of the lower passageway when the handle is in the open position.

8. The saliva ejector device of claim 3, wherein the upper and lower housings collectively form a substantially uniform exterior surface when in the closed position and the open position.

9. The saliva ejector device of claim 1, further comprising:
an automatic switching mechanism supported in the housing, the automatic switching mechanism comprising:
a hollow compliant tube positioned in the passageway and supported about an interior portion of the housing; and
a pressure-sensing operating switch disposed within a wall of the housing, and movable between a closed position and an open position upon application and release of pressure, respectively, thereon,
wherein, in the closed position, the pressure-sensing operating switch compresses the hollow compliant tube to terminate airflow through the housing, and in the open position, the pressure-sensing operating switch facilitates the expansion of the hollow compliant tube to initiate airflow through the housing.

10. The saliva ejector device of claim 9, in combination with a saliva ejector system comprising:
a vacuum conduit coupled to the saliva ejector device, and in fluid communication with the passageway in the housing and a vacuum source for facilitating airflow through the saliva ejector device;
a cradle having a receiving surface for interfacing with a surface of the housing, wherein upon insertion of the saliva ejector device in the cradle, the surface of the cradle automatically applies the pressure to the pressure-sensing operating switch sufficient to depress and activate the pressure-sensing operating switch to the closed position.

11. The saliva ejector device of claim 1, further comprising:
an automatic switching mechanism supported in the housing, the automatic switching mechanism comprising:
a pressure-sensing operating switch disposed within a wall of the housing, and movable between a closed position and an open position upon application and release of pressure, respectively, thereon, the pressure-sensing operating switch comprising a first end and a second end having a gate portion; and
a compliant device supported between the housing and the pressure-sensing operating switch to bias the pressure-sensing operating switch in an open position,
wherein, in the closed position, the gate portion engages the housing, such that the pressure-sensing operating switch terminates airflow through the housing, and in the open position, the pressure-sensing operating switch is disengaged from the housing to facilitate airflow through the housing.

12. The saliva ejector device of claim 11, in combination with a saliva ejector system comprising:
a vacuum conduit coupled to the saliva ejector device, and in fluid communication with the passageway in the housing and a vacuum source for facilitating airflow through the saliva ejector device;
a cradle having a receiving surface for interfacing with a surface of the housing, wherein upon insertion of the saliva ejector device in the cradle, the receiving surface of the cradle automatically applies the pressure to the pressure-sensing operating switch sufficient to depress and activate the pressure-sensing operating switch to the closed position.

13. A saliva ejector adapter operable for use with a handle of a saliva ejector device operating within a saliva ejector system, the saliva ejector adapter comprising:
a handle adapter comprising a body section having an ergonomic outer surface configuration for facilitating placement of a user's hand in a functional position upon grasping the handle;
a saliva ejector conduit interconnect formed about an upper opening of the handle adapter, the saliva ejector conduit interconnect having a configuration that facilitates coupling of the handle adapter to a saliva ejector conduit;
a handle interconnect formed about a lower opening of the handle adapter, the handle interconnect having a configuration that facilitates coupling of the handle adapter to a handle of a saliva ejector device; and
a passageway extending through the handle adapter and in fluid communication with the upper opening and the lower opening.

14. The saliva ejector adapter of claim 13, in combination with a saliva ejector system comprising:
a saliva ejector conduit;
a saliva ejector device having a handle, the handle having a saliva ejector conduit interconnect formed about an upper opening in the handle, and a vacuum conduit interconnect formed about a lower opening in the handle;
a vacuum conduit coupled to the vacuum conduit interconnect of the handle of the saliva ejector device, and in fluid communication with a vacuum source,
wherein the handle adapter is coupled to the handle of the saliva ejector device via the handle interconnect of the handle adapter and the saliva ejector conduit interconnect of the saliva ejector device, and
wherein the saliva ejector conduit is coupled to the handle adapter via the saliva ejector conduit interconnect of the handle adapter.

15. The saliva ejector adapter of claim 14, further comprising an automatic switching mechanism operable within the handle adapter, the automatic switching mechanism having an open position that facilitates airflow through the passageway and a closed position that restricts airflow about the passageway.

16. The saliva ejector adapter of claim 14, wherein the body section of the handle adapter is spherical in shape.

17. A saliva ejector suction system o ejecting saliva, comprising:
a vacuum device;
a vacuum conduit coupled to the vacuum device;
a cradle; and
a saliva ejector device coupled to the vacuum conduit, the saliva ejector device comprising a handle configured to be supported by the cradle, the handle comprising:
a saliva ejector conduit interconnect configured to receive and couple a saliva ejector conduit, the saliva ejector conduit interconnect having an opening there through;
a vacuum conduit interconnect configured to receive and couple the vacuum conduit, the vacuum conduit interconnect having an opening there through;
a passageway operable to establish fluid communication between the opening of the saliva ejector conduit interconnect and the opening of the vacuum conduit interconnect;
a body section comprising an ergonomic outer surface extending away from the saliva ejector conduit interconnect and the vacuum conduit interconnect a direction and distance so as to facilitate placement of a user's hand in a functional position upon grasping the handle.

18. The saliva ejector suction system of claim 17, wherein the ergonomic outer surface of the body section of the handle comprises a spherical configuration, and wherein the cradle comprises a spherical surface configuration operable to support and interface with the body section of the handle.

19. The saliva ejector suction system of claim 17, wherein the housing comprises:
an upper housing having an upper passageway formed therein to facilitate airflow through the upper housing; and
a lower housing having a lower passageway formed therein to facilitate airflow through the lower housing,
wherein the upper housing is rotatably coupled to the lower housing to selectively switch the handle between an open position with the upper passageway in fluid communication with the lower passageway to facilitate airflow through the handle, and a closed position with the upper passageway out of fluid communication with the lower passageway to restrict airflow through the handle.

20. The saliva ejector suction system of claim 19, wherein the upper housing and the lower housing each comprise a hemispherical shape and define, at least in part, the body section of the housing, such that the ergonomic outer surface of the body section of the housing comprises a spherical shape.

21. The saliva ejector suction system of claim 19, wherein the upper passageway comprises an offset passageway portion positioned offset relative to a central axis of the handle, and wherein the lower passageway comprises an offset passageway portion positioned offset relative to the central axis of the handle.

22. The saliva ejector suction system of claim 19, wherein, when in the open position, at least a portion of the first and second passageways are positioned offset relative to a central axis of the handle.

23. The saliva ejector suction system of claim 21, wherein the offset passageway portion of the upper passageway is in fluid communication with the offset passageway portion of the lower passageway when the handle is in the open position.

24. A method for making a saliva ejector device, comprising:
configuring a handle of a saliva ejector device to comprise:
a saliva ejector conduit interconnect configured to receive and couple a saliva ejector conduit, the saliva ejector conduit interconnect having an opening there through;
a vacuum conduit interconnect configured to receive and couple a vacuum conduit, the vacuum conduit interconnect having an opening there through;
a passageway operable to establish fluid communication between the opening of the saliva ejector conduit interconnect and the opening of the vacuum conduit interconnect;
a body section comprising an ergonomic outer surface extending away from the saliva ejector conduit interconnect and the vacuum conduit interconnect a direction and distance so as to facilitate placement of a user's hand in a functional position upon grasping the handle.
